(19) United States
(12) Patent Application Publication
(10) Pub. No.: US 2009/0130730 A1
(43) Pub. Date: May 21, 2009

(54) YEAST FOR EXTRACTION OF LIPID-SOLUBLE COMPONENT, METHOD FOR PRODUCING THE SAME, COLOR-IMPROVING AGENT USING THE SAME AND METHOD FOR PRODUCING LIPID-SOLUBLE COMPONENT

(75) Inventors: Tooru Abe, Shizuoka (JP); Yutaka Mitani, Shizuoka (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Sapporo Brewaries Limited, Shibuya-ku (JP)

(21) Appl. No.: 12/096,093

(22) PCT Filed: Nov. 1, 2006
(86) PCT No.: PCT/JP2006/321867

§ 371 Date: Oct. 17, 2008

(30) Foreign Application Priority Data
Dec. 9, 2005 (JP) 2005-356764

(51) Int. Cl. C12P 7/26; C12N 1/16

(52) U.S. Cl. 435/148; 435/255.7; 435/255.1

(57) ABSTRACT

The production process for yeast for fat-soluble component extraction according to the invention comprises a breeding step wherein yeast having fat-soluble components to be extracted are bred, in such a manner that the pH of the medium decreases as growth proceeds, until falling below the limit of the breedable pH range.

### TECHNICAL FIELD

The present invention relates to yeast for fat-soluble component extraction containing fat-soluble components to be extracted, as well as to a process for its production, to a color improver employing it and to a process for production of fat-soluble components.

### BACKGROUND ART

Yeast-employing processes are known as processes for producing various chemical components without chemical synthesis. Chemical components including fat-soluble components are produced in yeast, with the chemical components accumulating in the cells. For example, Xanthophyllomyces dendrorhous produces the fat-soluble component astaxanthin, with astaxanthin accumulating in the cells. Astaxanthin is known to have high antioxidative power of 500 times that of vitamin E.

However, because yeast generally have a rigid cell wall it is difficult to extract the intracellular components that have accumulated in the cells. The aforementioned Xanthophyllomyces dendrorhous yeast have a particularly strong cell wall. Extraction of astaxanthin found among the intracellular components of Xanthophyllomyces dendrorhous is therefore exceedingly difficult.

Methods are known for treating yeast themselves or yeast cell walls by physical, chemical or biological means for extraction of such yeast intracellular components.

Known physical treatment methods include crushing of the yeast themselves using a roll mill, French press, homogenizer, bead mill, ultrasonic waves or the like. For example, Patent Document 1 describes a method of using a roll mill for pressurizing treatment of dry cells of Phaffia rhodozyma (asexual generation of Xanthophyllomyces dendrorhous).

As chemical treatment methods there are known methods of using acids or alkalis for disruption or weakening of yeast cell walls. For example, Patent Document 2 describes using an acid for chemical treatment of Phaffia rhodozyma with heating at approximately 80° C., for utilization of the astaxanthin in the cells. Also, Patent Document 3 describes chemical treatment using an acid and/or alkali in the culturing process for Phaffia rhodozyma yeast cells, after a growth limiting stage under conditions with limited nutrients.

Biological treatment methods using cytolytic enzymes are also known. For example, Patent Document 4 describes a method of using an enzyme capable of lysing Phaffia rhodozyma cell walls, to lyse the cell walls and allow extraction of astaxanthin present in the cells.

### Patent Document 1: JP-A-H08-257

### Patent Document 2: JP-A-H06-7153

### Patent Document 3: JP-A-H08-228765

### Patent Document 4: JP-A-H08-259

### DISCLOSURE OF THE INVENTION

### Problem to be Solved by the Invention

However, the physical treatment method using a roll mill described in Patent Document 1 mentioned above requires high pressure treatment of several tens to several hundred atmospheres, and the method is therefore of low practicality. Other physical treatment methods, such as treatment with a French press, require pressurization at from several hundred to several thousand atmospheres. Also, treatment with a homogenizer, bead mill or ultrasonic waves is time consuming. Because of these drawbacks of physical treatment methods, they have not been readily applicable in industry.

Furthermore, the chemical treatment methods described in Patent Documents 2 and 3 require neutralizing treatment after the acid or alkali addition. They also require steps such as washing out of the acid or alkali used for the chemical treatment or washing out of the salts produced by neutralizing treatment, and therefore complex procedures have been necessary. When heating is applied during the chemical treatment, energy and equipment are required for the heating, and therefore such methods have not been industrially applicable. Such chemical treatment methods have been limited to use in cases where the total amounts of fat-soluble components of the intracellular components are extracted for analysis.

Also, in the biological treatment method described in Patent Document 4, separate apparatuses and steps are necessary to produce the enzymes for cell wall lysis, and this method has therefore been difficult to implement industrially.

It is an object of the present invention to provide yeast for fat-soluble component extraction with weakened cell walls, that allow efficient extraction of fat-soluble components present among the intracellular components of yeast, and a process for the production thereof. The invention further provides a process for production of fat-soluble components, and a color improver employing the yeast for fat-soluble component extraction.

### Means for Solving the Problem

The production process for yeast for fat-soluble component extraction according to the invention comprises a breeding step wherein yeast having fat-soluble components to be extracted are bred, in such a manner that the pH of the medium decreases as growth proceeds, until falling below the limit of the breedable pH range.

When microorganisms such as yeast are bred, the medium is adjusted to a constant pH in order to breed the yeast within the breedable pH range. Thus, it has been common knowledge that the pH during breeding should be constantly controlled, and particularly that deviation of the medium pH toward the acidic end, which is an inferior growth environment, should be avoided. However, the present inventors have discovered, in contrast to this common knowledge, that if the pH of the medium is lowered as growth proceeds, until the pH of the medium falls below the minimum limit of the breedable pH range, the fat-soluble components of the yeast can be efficiently extracted. According to this production process, it is possible to produce yeast for fat-soluble component extraction having weakened cell walls. Moreover, it is possible to extract fat-soluble components among intracellular components of such yeast for fat-soluble component extraction by an ordinary extraction step, without carrying out the conventional treatments of disruption or weakening of the cell walls.

In the aforementioned breeding step, the pH of the medium is preferably lowered by production of an acidic group from nutrients metabolized as growth proceeds. That is, preferably the yeast metabolize nutrients to produce an acidic group which gradually lowers the pH of the medium. In this case, external addition of a pH regulator will not necessarily be required to lower the medium pH during the breeding step.

The acidic group produced from nutrients metabolized during breeding of the yeast is preferably at least one selected from the group consisting of SO₄²⁻, HSO₃⁻, NO₃⁻, PO₄³⁻, HPO₄²⁻, H₂PO₄⁻, PO₃⁻ and Cl⁻. Nutrients that produce such acidic groups include ammonia salts such as ammonium sulfate, ammonium nitrate, ammonium phosphate and ammonium chloride. These contain the element nitrogen as a yeast nutrient and therefore also serve as nitrogen sources. The acidic groups that can be produced from these ammonia salts are SO₄²⁻ and HSO₃⁻ from ammonium sulfate, NO₃⁻ from ammonium nitrate, PO₄³⁻, HPO₄²⁻, H₂PO₄⁻ and PO₃⁻ from ammonium phosphate and Cl from ammonium chloride.

Xanthophyllomyces dendrorhous may be used in the production process for yeast for fat-soluble component extraction according to the invention. In this case, it is possible to obtain the yeast Xanthophyllomyces dendrorhous for fat-soluble component extraction that contains the fat-soluble component astaxanthin in the yeast cells and has weakened cell walls.

The astaxanthin can be extracted from the obtained Xanthophyllomyces dendrorhous for fat-soluble component extraction. The red carotenoid astaxanthin is used as a red pigment for color improvement of aquicultured fish, egg yolk and the like. Also, because astaxanthin has a powerful antioxidant effect, it is being studied for use as a medically active component and therefore has a wide variety of potential uses. The fat-soluble components in the yeast for fat-soluble component extraction are not limited to astaxanthin, however.

The present invention provides yeasts for fat-soluble component extraction that are obtained by the production process described above. The yeasts for fat-soluble component extraction obtained by the production process of the invention have weakened cell walls. Thus, it is possible to extract fat-soluble components among the intracellular components by an ordinary extraction step, without carrying out the conventional treatments of disruption or weakening of the cell walls.

The production process for fat-soluble components according to the invention comprises an extraction step in which the fat-soluble components are extracted from the yeast for fat-soluble component extraction. According to the production process for fat-soluble components according to the invention it is possible for the fat-soluble components contained in the yeast for fat-soluble component extraction that have weakened cell walls, to be extracted and produced by the subsequent extraction step, without carrying out the conventional treatments of disruption or weakening of the cell walls.

The present invention further provides a color improver containing the aforementioned yeast for fat-soluble component extraction. The color improver may be used by addition to a feed for aquicultured fish or poultry, for example. As mentioned above, the cell walls of the yeast for fat-soluble component extraction are weakened. Consequently, a color improver comprising the yeast for fat-soluble component extraction, and particularly yeast for fat-soluble component extraction containing astaxanthin as a fat-soluble component, may be given to aquicultured fish or the like with their feed, to be efficiently absorbed into the bodies of the aquicultured fish or the like in order to improve flesh color, body or egg color.

### EFFECT OF THE INVENTION

According to the invention there are provided yeast for fat-soluble component extraction with weakened cell walls, that allow efficient extraction of fat-soluble components present among the intracellular components of the yeast, and a process for the production thereof. The invention also provides a process for production of fat-soluble components and a color improver employing the yeast for fat-soluble component extraction.

### BRIEF DESCRIPTION OF THE DRAWINGS

FIG. 1 is a graph showing the time-dependent change in medium pH and medium yeast concentration during the main culturing in Example 1.

FIG. 2 is a graph showing the time-dependent change in medium pH and medium yeast concentration during the main culturing in Comparative Example 4.

FIG. 3 is a graph showing the time-dependent change in medium pH and medium yeast concentration during the main culturing in Example 8.

FIG. 4 is a graph showing the time-dependent change in medium pH and medium yeast concentration during the main culturing in Comparative Example 5.

### BEST MODES FOR CARRYING OUT THE INVENTION

Embodiments of the invention will now be explained in detail.

A process for production of yeast for fat-soluble component extraction according to the invention will be explained first. The production process for yeast for fat-soluble component extraction according to the invention is characterized by comprising a breeding step wherein yeast having fat-soluble components to be extracted are bred in such a manner that the pH of the medium decreases as growth proceeds, until it falls below the limit of the breedable pH range (hereinafter referred to simply as “breeding step”).

The yeast used for breeding in the breeding step is a yeast containing fat-soluble components among the intracellular components. As examples there may be mentioned Xanthophyllomyces dendrorhous, Saccharomyces yeasts and Candida yeasts. These yeasts may have various mutations introduced therein for increased production of the fat-soluble components. For example, there may be used Xanthophyllomyces dendrorhous which is a high-producing mutant obtained by introduction of a mutation by the method described in Japanese Patent Public Inspection HEI No. 6-506122.

The method for culturing the yeast to breed the yeast in the breeding step may be a batch process, semi-batch process, feeding culture process or continuous culture process. These culturing processes and the culturing conditions may be selected as appropriate for the type of yeast and medium used.

The method of lowering the pH of the medium as growth proceeds may be a pH lowering method involving external addition of a small amount of acid, or a spontaneous pH lowering method based on the yeast metabolism. Spontaneous pH lowering methods include a method of spontaneously lowering the pH by acid produced by the yeast metabolism, and a method of spontaneously lowering the pH by production of acidic groups from metabolized nutrients. Of these methods, spontaneous lowering of the pH by production of acidic groups from metabolized nutrients is preferred. More specifically, this method achieves a lower medium pH because the components in the medium serving as nutrients for the yeast are metabolized and the acidic groups in the components are released into the medium. This method allows the medium pH to be sufficiently lowered to below the minimum limit of the breedable pH range as the yeast carry out metabolism. When a spontaneous pH lowering method based on yeast metabolism is used, the breeding step is preferably carried out under aerobic conditions to adequately promote yeast metabolism.

As nutrients that produce acidic groups by metabolism there are preferred ammonia salts that contain the element nitrogen as a yeast nutrient and produce acidic groups upon metabolism. As such ammonia salts there are preferred ammonium sulfate, ammonium nitrate, ammonium phosphate, ammonium chloride and the like. The acidic groups that can be produced from such ammonia salts include SO₄²⁻ and HSO₃⁻ from ammonium sulfate, NO₃⁻ from ammonium nitrate, PO₄³⁻, HPO₄²⁻, H₂PO₄⁻ and PO₃⁻ from ammonium phosphate and Cl⁻ from ammonium chloride.

In order to adequately lower the pH of the medium, the acidic groups produced by nutrient metabolism are preferably ones having a high dissociation degree in the medium solution. Among the aforementioned acidic groups there are preferred SO₄²⁻, NO₃⁻, Cl⁻ as acidic groups produced by nutrient metabolism, with SO₄²⁻ and NO₃⁻ being preferred.

The medium used for the breeding step may be a medium containing a malt extract diluent, a synthetic medium containing glucose and yeast extract, or a similar medium containing various additives. When the pH of the medium is spontaneously lowered as growth proceeds, it is preferred to add to the medium an ammonia salt such as ammonium sulfate, ammonium nitrate, ammonium phosphate or ammonium chloride, and more preferably ammonium sulfate.

The breedable pH range for the yeast having the fat-soluble components to be extracted may be determined by preparing multiple culturing media with different pH values, breeding the yeast while maintaining the pH values of the media, and determining the breeding ratio of the yeast in the medium at each pH value. The breedable pH range of the yeast obtained in this manner will differ depending on the yeast strain, but the range will normally be 4-9. That is, it is difficult to breed yeast in medium below the minimum limit breedable pH range, such as below 4. According to the invention, however, it is possible to produce yeast for fat-soluble component extraction that have weakened cell walls by allowing the pH to fall below the minimum limit of the breedable pH range for the yeast.

The fat-soluble components extracted from the yeast for fat-soluble component extraction may be carotenoids such as astaxanthin, fat-soluble vitamins such as vitamins A, D and E, sterols, terpenes, fat-soluble amino acids, fat-soluble proteins and the like. Among these fat-soluble components, the red carotenoid astaxanthin is used as a red pigment for color improvement of aquicultured fish, egg yolk and the like. Also, because astaxanthin has a powerful antioxidant effect, it is being studied for use as a medically active component. Astaxanthin can be extracted from Xanthophyllomyces dendrorhous produced by the aforementioned process as a yeast for fat-soluble component extraction.

Astaxanthin is widely distributed in nature, being found in crustacean shells and in eggs, salmon flesh, kinmedai epidermis and the like. Astaxanthin plays a role for their flesh color or body color. As microorganisms that produce astaxanthin there are also known, in addition to Xanthophyllomyces dendrorhous, also the algal species Haematococcus pluvialis.

According to the process for production of yeast for fat-soluble component extraction according to the invention, it is possible to produce yeast for fat-soluble component extraction with weakened cell walls.

In the process for production of yeast for fat-soluble component extraction according to the invention, it is sufficient to lower the pH of the medium to below the minimum limit of the breedable pH range in order to halt growth of the yeast for fat-soluble component extraction. That is, it is not necessarily required to breed the yeast with the pH of the medium being lowered as growth proceeds throughout the entire process of breeding the yeast. A conventionally known breeding method may be used for the initial stage of the yeast breeding, and the invention breeding step carried out at a latter stage of breeding. For example, the production process may employ culture scale-up means of an ordinary method for transfer from seed culture to submerged culture in a conventionally known medium, followed by a breeding step for the main culturing.

The yeast do not necessarily need to be grown constantly during the breeding step. The breeding step may include a non-yeast-growing culturing step under conditions with restricted growth nutrients for the purpose of yeast preservation, etc. A non-yeast-growing culturing step may also be carried out after completion of the breeding step.

The yeast for fat-soluble component extraction bred in the breeding step are then collected using a centrifugal separator or the like. The collected slurry of yeast for fat-soluble component extraction is preferably subjected to one or multiple water washing treatments as necessary to obtain a yeast cell slurry. Depending on the purpose of use, this may be combined with drying treatment to obtain wet yeast cells or dry yeast cells.

The yeast for fat-soluble component extraction obtained in this manner have weakened cell walls. Thus, it is possible to extract fat-soluble components among the intracellular components by an ordinary extraction step, without carrying out the conventional treatments of disruption or weakening of the cell walls.

The yeast for fat-soluble component extraction of the invention is preferably Xanthophyllomyces dendrorhous for extraction of fat-soluble components, including astaxanthin as a fat-soluble component. Since the Xanthophyllomyces dendrorhous for extraction of fat-soluble components has weakened cell walls, not only is the astaxanthin efficiently extracted, but its features permit use for other purposes as well. For example, it may be used as a color improver, nutritional supplement, external skin application, immunoenhancer, ocular disease ameliorator or the like. Because the Xanthophyllomyces dendrorhous for extraction of fat-soluble components has weakened cell walls, the astaxanthin can be efficiently absorbed into the body when the Xanthophyllomyces dendrorhous is given together with animal feeds, food products and the like. It is therefore possible to fully utilize the color improving effects of astaxanthin to improve flesh color, body color or egg color, as well as its antioxidant effects.

The fat-soluble component production process of the invention will now be explained. The production process for fat-soluble components according to the invention comprises an extraction step in which the fat-soluble components are extracted from the aforementioned yeast for fat-soluble component extraction.

The yeast for fat-soluble component extraction have weakened cell walls. Thus, it is possible to extract and produce fat-soluble components among the intracellular components by a subsequent extraction step, without carrying out the conventional treatments of disruption or weakening of the cell walls.

In the fat-soluble component extraction step, an organic solvent is used to extract the fat-soluble components from the yeast for fat-soluble component extraction which is in a yeast cell slurry or wet yeast cell state. As organic solvents there may be used acetone, ethanol, hexane, chloroform or the like, or mixtures thereof. The fat-soluble components extracted into the organic solvent may be recovered by evaporation of the organic solvent using an evaporator or the like.

The color improver according to the invention will now be explained. The color improver of the invention comprises the yeast for fat-soluble component extraction. As mentioned above, the cell walls of the yeast for fat-soluble component extraction are weakened. Consequently, a color improver comprising the yeast for fat-soluble component extraction may be given to aquicultured fish, poultry or the like with their feed, to be efficiently absorbed into the bodies of the aquicultured fish or the like in order to improve flesh, body or egg color.

The color improver may consist entirely of the yeast for fat-soluble component extraction, but it may also comprise a mixture of the yeast for fat-soluble component extraction with dietary fiber, oligosaccharides, polysaccharides and the like. Also, vitamins, minerals and the like may also be included to simultaneously provide functions as nutritional supplements in addition to the color improving effect.

### EXAMPLES

The present invention will now be explained in greater detail using examples, with the understanding that the invention is in no way limited to the examples. Throughout the following examples and comparative examples, a culture solution pH value of 4 was defined as the minimum limit of the breedable pH range for the yeast.

### Example 1

### Culturing of Xanthophyllomyces dendrorhous

For scale-up of Xanthophyllomyces dendrorhous cells (ATCC24202) preserved in slant medium, the ATCC24202 cells were pre-cultured in malt extract diluent medium with a 6 wt % sugar content prior to main culturing. That is, the cells were inoculated from slant medium for culturing at 20° C. for 48 hours, to obtain a pre-culture solution.

As the medium for main culturing there was prepared in an Erlenmeyer flask (500 ml volume) 90 ml of medium containing 0.3 wt % ammonium sulfate added to malt extract diluent with a 6 wt % sugar content. A 10 ml portion of the culture solution obtained from the pre-culturing was seeded in the main culture medium. The main culturing was shake cultured at 180 rpm, 20° C. for 144 hours.

<Extraction of Astaxanthin>

Upon completion of the main culturing, 100 ml of medium was placed in a centrifugal separator to recover the Xanthophyllomyces dendrorhous yeast cells (hereinafter, “yeast cells”) in the medium. The recovered yeast cells were rinsed twice with water to obtain a yeast cell slurry for extraction of astaxanthin.

After placing 100 mg of the yeast cell slurry in an Erlenmeyer flask, 8 ml of acetone was added. The Erlenmeyer flask was situated in a cool dark space at 5° C. for 3 hours, and the astaxanthin in the yeast cell components was extracted into acetone.

<Measurement of Astaxanthin Extract Weight>

After standing in a cool dark space for 3 hours, the weight of astaxanthin floating on the acetone liquid surface was measured using an absorptiometer. The weight of the astaxanthin measured in this manner was divided by the weight of the Xanthophyllomyces dendrorhous dry yeast cells used for extraction (hereinafter, “dry yeast cells”) to obtain the astaxanthin extract weight per unit weight of dry yeast cells as the astaxanthin extract yield (μg/g). The dry yeast cell weight was calculated from the results of measuring the yeast cell concentration in the medium upon completion of the main culturing. Measurement of the yeast cell concentration in the medium was conducted as follows.

<Measurement of Yeast Cell Concentration in Medium>

The yeast cell concentration in the medium was measured during the main culturing, immediately after starting the main culturing and 24 hr, 48 hr, 72 hr, 96 hr, 120 hr and 144 hr after starting the main culturing. The weight of dry yeast cells in the medium per unit volume was recorded as the yeast cell concentration of the medium (g/l).

At each of the aforementioned time points during the main culturing there was added 30 ml of yeast cell-containing medium. The sampled medium was placed in a centrifugal separator for recovery of the yeast cells in the medium. The recovered yeast cells were placed in a thermostatic bath at 105° C. for 24 hours for drying. The weight of the dry yeast cells obtained in this manner was measured, and the value was divided by the volume of the sampled medium to calculate the yeast cell concentration of the medium.

<Medium pH Measurement>

The pH of the medium was measured during the main culturing, immediately after starting the main culturing and 24 hr, 48 hr, 72 hr, 96 hr, 120 hr and 144 hr after starting the main culturing. The medium pH was measured using a glass electrode-type hydrogen ion content meter.

### Comparative Example 1

Main culturing was conducted in the same manner as Example 1, except that ammonia water was gradually added to the medium to maintain a medium pH of 4.5, for culturing of the yeast cells, extraction of astaxanthin and measurement of the astaxanthin extract yield.

### Comparative Example 2

Main culturing was conducted in the same manner as Example 1, except that a malt extract diluent with a 6 wt % sugar content was used and no ammonium sulfate was added to the medium, for culturing of the yeast cells, extraction of astaxanthin and measurement of the astaxanthin extract yield.

### Comparative Example 3

Cell wall disruption treatment with an acid was carried out for extraction of astaxanthin from yeast cells. Specifically, the yeast cells were cultured with gradual addition of ammonia water to the medium to maintain a medium pH of 4.5, in the same manner as Comparative Example 1. The yeast cell slurry obtained in this manner was placed in an Erlenmeyer flask and dilute sulfuric acid was added to 1N concentration. The Erlenmeyer flask was immersed in a boiling water bath, and after 5 minutes of boiling it was rapidly cooled. The cell wall-disrupted yeast cells were recovered using a centrifugal separator. The recovered yeast cells were rinsed twice with water to obtain a yeast cell slurry for extraction of astaxanthin. After obtaining the yeast cell slurry, the astaxanthin was extracted and the astaxanthin extract yield was measured in the same manner as Example 1.

### Comparative Example 4

Lactic acid was added to the medium during the main culturing to abruptly lower the medium pH, so that the pH at completion of culturing was below the minimum limit of the breedable pH range. Specifically, pH adjustment of the medium by lactic acid addition was initiated 48 hours after start of the main culturing, and a medium pH of 2.2 was maintained. The main culturing was stopped 32 hours after initiating lactic acid addition.

Comparative Example 4 also differed from Example 1 in the following aspects. Specifically, the medium used for the main culturing was medium containing 0.3 wt % ammonium sulfate and 1000 ppm calcium carbonate with respect to malt extract diluent with a 6 wt % sugar content. Calcium carbonate was added to moderate the rate of pH lowering by growth of the yeast cells, in order to ensure a sufficient period for yeast cell growth. After main culturing under these conditions, the astaxanthin was extracted and the astaxanthin extract yield was measured in the same manner as Example 1.

The results of measuring the astaxanthin extract yields for Example 1 and Comparative Examples 1-4 are shown in Table 1. Table 1 also shows the medium pH values and medium yeast cell concentrations at the start and end of the main culturing.

TABLE 1

| | | | Comp. | Comp. | Comp. | Comp. |
|---|---|---|---|---|---|---|
| | | Exam- | Exam- | Exam- | Exam- | Exam- |
| | | ple 1 | ple 1 | ple 2 | ple 3 | ple 4 |
| Culture | Start of | 4.8 | 4.5 | 5.5 | 4.5 | 6.7 |
| medium pH | main | | | | | |
| | culturing | | | | | |
| | End of | 2.2 | 4.5 | 4.0 | 4.5 | 2.3 |
| | main | | | | | |
| | culturing | | | | | |
| Yeast cell | Start of | 1.2 | 0.5 | 0.4 | 0.5 | 0.4 |
| concen- | main | | | | | |
| tration | culturing | | | | | |
| of medium | End of | 12.7 | 11.4 | 12.7 | 11.4 | 8.0 |
| (g/l) | main | | | | | |
| | culturing | | | | | |

| | | | | | |
|---|---|---|---|---|---|
| Astaxanthin extract | 271 | 63 | 79 | 258 | 59 |
| yield (μg/g) | | | | | |

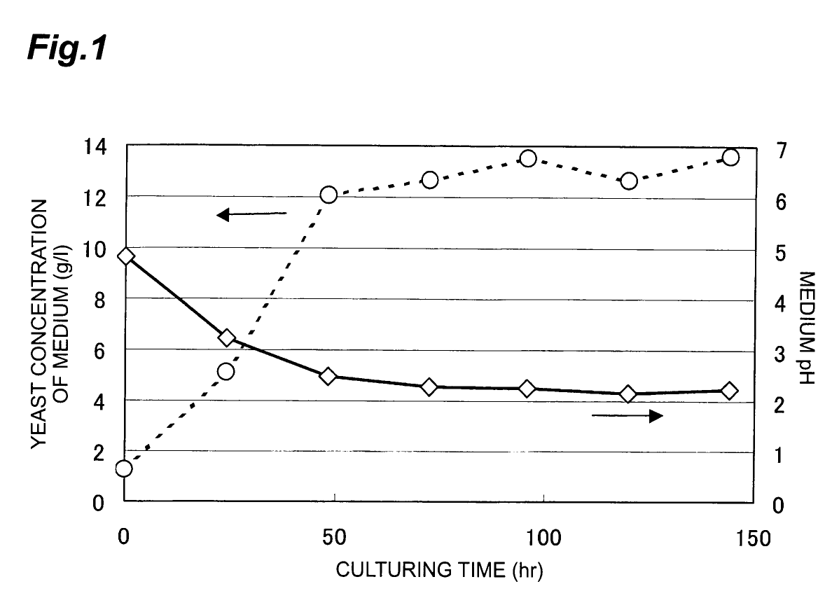

FIG. 1 is a graph showing the time-dependent change in medium pH and medium yeast concentration during the main culturing in Example 1. An increase in medium yeast concentration indicates growth of yeast cells. In FIG. 1, growth of yeast cells occurs rapidly at 48 hours after start of the main culturing. The medium pH decreases to about 2.5 as yeast cell growth proceeds. This value is well below 4, the minimum limit of the breedable pH range for yeast. Thus, growth of the yeast cells was inhibited, and lowering of the medium pH was also inhibited, from the stage 48 hours after the start of the main culturing.

In Comparative Example 1, however, a medium pH of 4.5 within the breedable pH range was maintained throughout the main culturing. As shown in Table 1, even in Comparative Example 1 the yeast cells grew based on comparison between the yeast cell concentrations at the start and end of the main culturing. However, since the pH of the medium did not fall as yeast cell growth proceeded, the astaxanthin extract yield was lower than in Example 1.

In Comparative Example 2, the medium pH was not kept constant, but since no ammonium sulfate was present in the medium, the level of acidic groups metabolized with proceeding yeast growth was insufficient. Consequently, the medium pH at the end of the main culturing was insufficiently lowered, and remained at 4.0 within the breedable pH range.

In Comparative Example 3, on the other hand, yeast cell wall disruption treatment was accomplished with an acid after the main culturing carried out in the same manner as Comparative Example 1, and therefore the astaxanthin extract yield was relatively high. This cell wall disruption treatment with an acid is commonly carried out for extraction of the total astaxanthin in cell components to be used in analysis, but cell wall disruption treatment is not industrially suitable. For Comparative Example 3, steps prior to astaxanthin extraction are necessary, such as the acid cell wall disruption treatment, alkali neutralization step and the washing step for the salt produced by neutralization, and therefore astaxanthin could not be efficiently extracted.

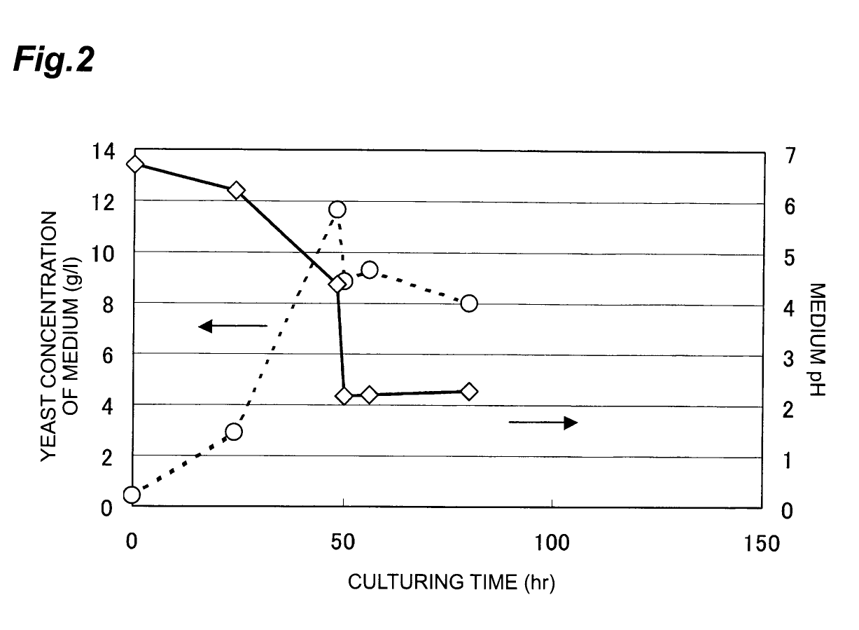

FIG. 2 is a graph showing the time-dependent change in medium pH and medium yeast concentration during the main culturing in Comparative Example 4. In FIG. 2, growth of yeast cells occurs rapidly at 48 hours after start of the main culturing, as in FIG. 1. The medium pH decreases to about 4.4 as yeast cell growth proceeds. This value is within the breedable pH range for yeast, and is relatively high compared to the pH of 2.5 at the same time point in Example 1. This is attributed to the effect of the calcium carbonate added to the medium. This demonstrates that using lactic acid to rapidly lower the medium pH externally from the breedable pH range cannot produce the high astaxanthin extract yield of Example 1.

Incidentally, since the astaxanthin extract yield differs minutely depending on the culturing batch, the main culturing and measurements were conducted twice in the same manner for Example 1 and Comparative Examples 1 and 3, and this produced the same results for Example 1 and Comparative Examples 1 and 3.

### Example 2

Culturing of yeast cells, extraction of astaxanthin and measurement of the astaxanthin extract yield were conducted in the same manner as Example 1, except that the medium for the main culturing was a medium containing 0.3 wt % of ammonium nitrate instead of ammonium sulfate added with respect to the malt extract diluent with a 6 wt % sugar content.

### Example 3

Culturing of yeast cells, extraction of astaxanthin and measurement of the astaxanthin extract yield were conducted in the same manner as Example 1, except that the medium for the main culturing was a medium containing 0.3 wt % of ammonium chloride instead of ammonium sulfate added with respect to the malt extract diluent with a 6 wt % sugar content.

### Example 4

Culturing of yeast cells, extraction of astaxanthin and measurement of the astaxanthin extract yield were conducted in the same manner as Example 1, except that the medium for the main culturing was a medium containing 0.3 wt % of ammonium phosphate instead of ammonium sulfate added with respect to the malt extract diluent with a 6 wt % sugar content.

The results of measuring the astaxanthin extract yields for Examples 2 to 4 are shown in Table 2. Table 2 also shows the medium pH values and medium yeast cell concentrations at the start and end of the main culturing.

TABLE 2

| | | Example 2 | Example 3 | Example 4 |
|---|---|---|---|---|
| Ammonia salt in medium | | Ammonium | Ammonium | Ammonium |
| | | nitrate | chloride | phosphate |
| Culture medium | Start of | 4.4 | 4.4 | 4.4 |
| pH | main | | | |
| | culturing | | | |
| | End of | 2.3 | 2.0 | 3.9 |
| | main | | | |
| | culturing | | | |
| Yeast cell | Start of | 0.9 | 0.9 | 0.9 |
| concentration of | main | | | |
| medium (g/l) | culturing | | | |
| | End of | 5.2 | 10.4 | 17.2 |
| | main | | | |
| | culturing | | | |

| | | | |
|---|---|---|---|
| Astaxanthin extract yield | 313 | 305 | 97 |
| (μg/g) | | | |

As shown in Table 2, the astaxanthin extract yields for Examples 2 and 3 were greater than 300 μg/g, and therefore high extract yields were obtained. The astaxanthin extract yield for Example 4, wherein the ammonia salt in the medium was ammonium phosphate, was higher than Comparative Example 2 which used a medium containing no ammonia salt, but the value was still only about 100 μg/g. The reason for this is believed to be that the acidic groups produced by metabolism of ammonium phosphate, namely PO₄³⁻, HPO₄²⁻, H₂PO₄⁻ and PO₃⁻, have relatively low dissociation degrees in the medium, and therefore the medium pH was not sufficiently lowered in comparison to adding ammonia salts such as ammonium sulfate, ammonium nitrate and ammonium chloride. Still, the phosphorus in ammonium phosphate has a favorable effect on yeast cell growth, and the yeast cell concentration of the medium at the end of main culturing was as high as 17.2 g/l. Thus, ammonium phosphate may be suitably added to the medium in combination with ammonia salts such as ammonium sulfate, ammonium nitrate and ammonium chloride. This can potentially achieve a high level of both yeast cell concentration and astaxanthin extract yield even with the same main culturing method as in Example 4.

### Example 5

Culturing of yeast cells, extraction of astaxanthin and measurement of the astaxanthin extract yield were conducted in the same manner as Example 1, except that the medium for the main culturing was a synthetic medium containing 0.3 wt % ammonium sulfate instead of the malt extract diluent with a 6 wt % sugar content. The composition of the synthetic medium is shown in Table 3.

TABLE 3

| Component | Concentration (g/l) |
|---|---|
| Glucose | 40 |
| Yeast extract | 4 |
| Ammonium sulfate | 3 |
| Potassium hydrogenphosphate | 1 |
| Magnesium sulfate heptahydrate | 0.5 |
| Calcium chloride dihydrate | 0.1 |

### Example 6

Culturing of yeast cells, extraction of astaxanthin and measurement of the astaxanthin extract yield were conducted in the same manner as Example 1, except that the Xanthophyllomyces dendrorhous strain used was a high astaxanthin-producing mutant instead of ATCC24202.

The results of measuring the astaxanthin extract yields for Examples 5 and 6 are shown in Table 4. Table 4 also shows the medium pH values and medium yeast cell concentrations at the start and end of the main culturing.

TABLE 4

| | | Example 5 | Example 6 |
|---|---|---|---|
| Culture medium pH | Start of main | 4.9 | 5.2 |
| | culturing | | |
| | End of main | 2.2 | 2.3 |
| | culturing | | |
| Yeast cell | Start of main | 1.0 | 0.9 |
| concentration of | culturing | | |
| medium (g/l) | End of main | 7.2 | 10 |
| | culturing | | |

| | | |
|---|---|---|
| Astaxanthin extract yield (μg/g) | 571 | 315 |

### Example 7

In order to accomplish main culturing using a culturing apparatus capable of submerged culture, the Xanthophyllomyces dendrorhous strain ATCC24202 preserved in slant medium was scaled-up in two stages. After inoculating the cells from the slant medium to a medium containing malt extract diluent with a 6 wt % sugar content, they were cultured at 20° C. for 48 hours as a first pre-culturing.

As a medium for the second pre-culturing there was prepared 90 ml of malt extract diluent medium with a 6 wt % sugar content in a 500 ml Erlenmeyer flask. Two such Erlenmeyer flasks were prepared. A 10 ml portion of culture solution obtained from the first pre-culturing was seeded in the medium in each Erlenmeyer flask. The second pre-culturing was shake cultured at 180 rpm, 20° C. for 48 hours.

As the medium for the main culturing there was prepared 1.8 liters of medium containing 0.3 wt % ammonium sulfate added with respect to a malt extract diluent with a 6 wt % sugar content, in the culture tank of a culturing apparatus (5 liter volume). The culturing apparatus was equipped with a temperature-control device and jar fermentor. After adding 180 ml of the culture solution obtained by the second pre-culturing to the culture solution in the culturing apparatus, submerged culture was conducted at 20° C. for 144 hours.

Upon completion of the main culturing, the astaxanthin was extracted and the astaxanthin extract yield was measured in the same manner as Example 1.

### Example 8

For main culturing, the medium pH was adjusted to 4.5 until the sugar concentration of the medium fell below 3%. Adjustment of the medium pH to 4.5 was accomplished by slow addition of ammonia water to the medium. The sugar concentration of the medium fell below 3% at 48 hours after start of the main culturing. The pH of the medium was not adjusted thereafter. The yeast cells were cultured in the same manner as Example 7, except for this adjustment of the medium pH in the main culturing. Upon completion of the main culturing, the astaxanthin was extracted and the astaxanthin extract yield was measured in the same manner as Example 1. Measurement of the sugar concentration of the medium was accomplished by HPLC.

### Comparative Example 5

Yeast cells were cultured in the same manner as Example 7, except that in the main culturing, ammonia water was slowly added to the medium to keep the medium pH at 4.5. Upon completion of the main culturing, the astaxanthin was extracted and the astaxanthin extract yield was measured in the same manner as Example 1.

The results of measuring the astaxanthin extract yields for Examples 7 and 8 and Comparative Example 5 are shown in Table 5. Table 5 also shows the medium pH values and medium yeast cell concentrations at the start and end of the main culturing.

TABLE 5

| | | | | Comp. |
|---|---|---|---|---|
| | | Example 7 | Example 8 | Example 5 |
| Culture medium | Start of main | 4.3 | 4.3 | 4.5 |
| pH | culturing | | | |
| | End of main | 2.6 | 2.4 | 4.5 |
| | culturing | | | |
| Yeast cell | Start of main | 1.8 | 1.8 | 7.8 |
| concentration of | culturing | | | |
| medium (g/l) | End of main | 6.9 | 9.1 | 14 |
| | culturing | | | |

| | | | |
|---|---|---|---|
| Astaxanthin extract yield (μg/g) | 230 | 263 | 63 |

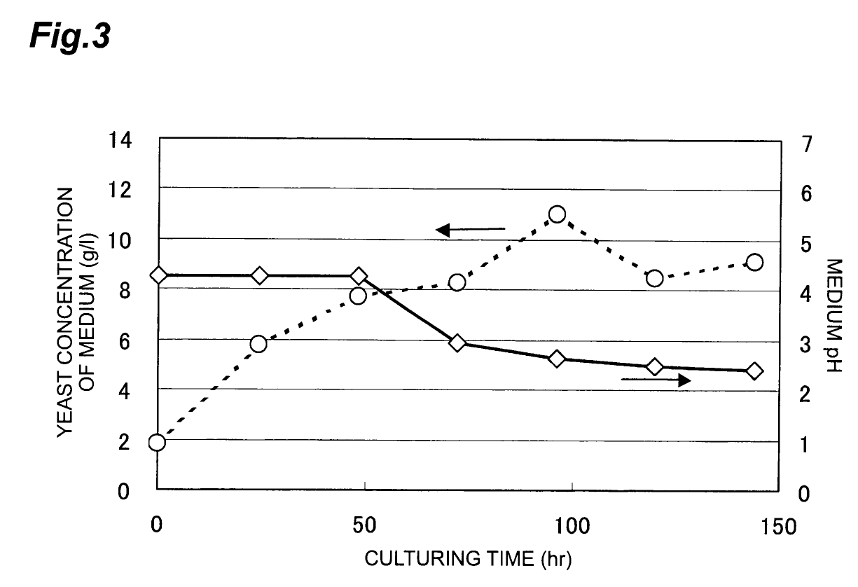

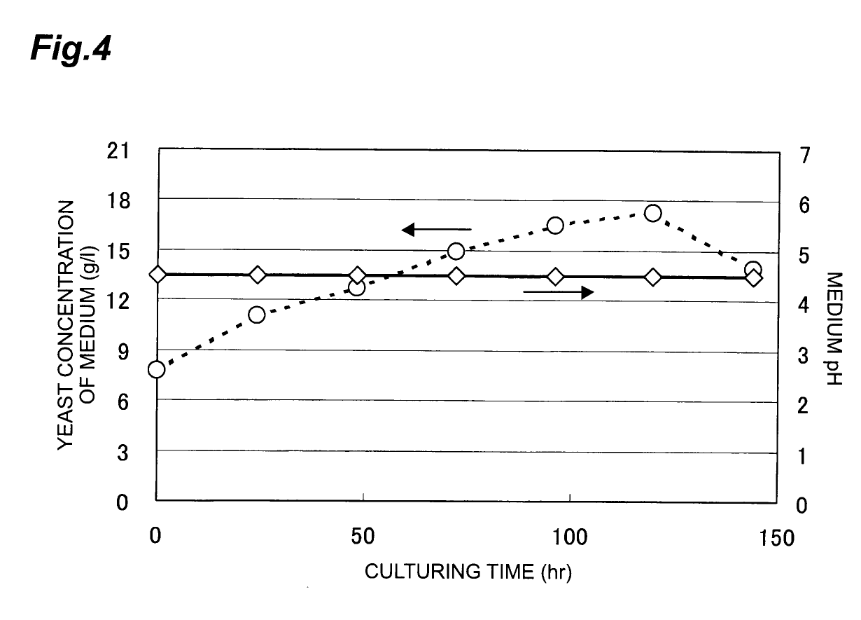

FIG. 3 is a graph showing the time-dependent change in medium pH and medium yeast concentration during the main culturing in Example 8. FIG. 4 is a graph showing the time-dependent change in medium pH and medium yeast concentration during the main culturing in Comparative Example 5.

In Example 8, as shown in FIG. 3, the yeast cell growth was satisfactory for 48 hours after the start of main culturing during which the medium pH was adjusted. The yeast cells continued to grow for about 48 hours after the medium pH was no longer adjusted, with the medium pH falling as growth proceeded and eventually reaching a value of about 2.5. This value is well below 4, the minimum limit of the breedable pH range for yeast.

Upon comparing the astaxanthin extract yields of Examples 7 and 8, that of Example 8 was found to be higher. It is believed that adequate yeast cell growth could be achieved in Example 8 because the medium pH in the initial stage of the main culturing was within the breedable pH range for yeast.

On the other hand, in Comparative Example 5 in which the main culturing was carried out while maintaining the medium pH at 4.5, which is within the breedable pH range, the yeast cells grew for 120 hours from the start of the main culturing, as shown in FIG. 4. However, since the pH of the medium did not fall as yeast cell growth proceeded, the astaxanthin extract yield was lower than in Examples 7 and 8.

### INDUSTRIAL APPLICABILITY

According to the invention there are provided yeast for fat-soluble component extraction with weakened cell walls, that allow efficient extraction of fat-soluble components present among the intracellular components of the yeast, and a process for the production thereof. The invention also provides a process for production of fat-soluble components and a color improver employing the yeast for fat-soluble component extraction.

1. A production process for yeast for fat-soluble component extraction, comprising breeding yeast having a fat-soluble component to be extracted, wherein a pH of a medium decreases as growth proceeds, until falling below the limit of the breedable pH range.

2. The production process for yeast for fat-soluble component extraction according to claim 1, wherein in the breeding step, the pH of the medium decreases by production of an acidic group from a nutrient that is metabolized as growth proceeds.

3. The production process for yeast for fat-soluble component extraction according to claim 2, wherein the acidic group is at least one selected from the group consisting of SO₄²⁻, HSO₃⁻, NO₃⁻, PO₄³⁻, HPO₄²⁻, H₂PO₄₋, PO₃⁻ and Cl⁻.

4. The production process for yeast for fat-soluble component extraction according to claim 1, wherein the yeast is Xanthophyllomyces dendrorhous.

5. The production process for yeast for fat-soluble component extraction according to claim 1, wherein the fat-soluble component is astaxanthin.

6. Yeast for fat-soluble component extraction that is produced by the production process according to claim 1.

7. A production process for a fat-soluble component, comprising extracting a fat-soluble component from the yeast for fat-soluble component extraction produced by the production process according to claim 1.

8. A color improver comprising the yeast for fat-soluble component extraction according to claim 6.
